(19) United States
(12) Patent Application Publication
(10) Pub. No.: US 2012/0029392 A1
(43) Pub. Date: Feb. 2, 2012

(54) METHOD AND SYSTEM FOR DETECTING A FALL OF A USER

(75) Inventors: Sheng Jin, Shanghai (CN); Ningjiang Chen, Shanghai (CN)

(73) Assignee: KONINKLIJKE PHILIPS ELECTRONICS N.V., EINDHOVEN (NL)

(21) Appl. No.: 13/260,949

(22) PCT Filed: Mar. 26, 2010
(86) PCT No.: PCT/IB2010/051325

§ 371 Date: Sep. 29, 2011

(30) Foreign Application Priority Data
Apr. 3, 2009 (CN) 200910134102.1

(51) Int. Cl. A61B 5/11

(52) U.S. Cl. 600/595

(57) ABSTRACT

This invention relates to a system and method for detecting a fall of a user. The system comprises at least one sensor, a determining unit and a processor. The at least one sensor worn on the body of the user generates sensor data relating to the fall, and the determining unit determines a sensor position of the at least one sensor. The processor adjusts the fall detection algorithm according to the sensor position, and performs an analysis based on the sensor data to determine whether a fall is occurring or not. In this way, fall detection is performed with high detection accuracy even if the sensor position changes, and the user feels good by wearing the sensors in their preferred way.

### FIELD OF THE INVENTION

The invention relates to a method and system for detecting a fall of a user.

### BACKGROUND OF THE INVENTION

Many people are at increased risk of injury or death as a result of a chronic health condition or complications resulting from acute illness, disability, or advancing age. Many other people suffer from chronic, or at least sustained, conditions that require long term treatment. Other people, such as soldiers, police, fire fighters, rescue workers, etc., work under hazardous and life-threatening conditions. In many instances, detecting a fall of these individuals is necessary to render aid when needed to prevent further health issues that could result from a fall.

To detect a fall of a user, different kinds of sensors are applied, such as an accelerometer, an altimeter, a thermometer, etc. These sensors measure different kinds of parameters, such as acceleration, gravity, altitude, temperature, etc., and then detect whether a fall is occurring or not, based on the measured parameters. Sensors are usually positioned on the body of the user to measure a number of parameters. For different positions of the sensor(s) relative to the body, the probabilities of correctly detecting a fall are different. For example, when an altimeter is positioned on the body for fall detection, the probability of correctly detecting a fall when the altimeter is worn around the neck is higher than when the altimeter is worn on a wrist. This can be attributed to the fact that the wrist moves up and down more frequently than the neck when no fall occurs, and the altitude change of the neck is larger than that of the wrist when a fall occurs. Therefore, for fall detection, sensors are usually worn around the neck to improve detection accuracy.

US2006/0282021 discloses a motion analysis telemonitor system including a wearable monitoring device that monitors the activity level and movements of a person wearing the device. The wearable monitoring device is able to determine whether the person has fallen by means of a model analysis technique using characteristic movements of a fall. The wearable device generally transmits data and alerts over a short distance to a console. The console, in turn, transmits data and alerts to a monitoring centre. The motion analysis telemonitor system is also able to monitor progression of disease through changes in movement, as well as fatigue and other performance factors.

### SUMMARY OF THE INVENTION

Most of the current solutions for detecting a fall work by placing the sensors in a best position on the body, such as the neck, to improve the detection accuracy. However, inventors of this patent application have found that only placing the sensors in the best position for high detection accuracy cannot fulfill the users' requirements.

Taking fall detection of elderly users as an example, it is better for the elderly users to wear the sensors, attached to a band, around the neck to improve fall detection accuracy. However, the best position with high detection accuracy during the daytime becomes the worst position making the users uncomfortable during the nighttime. During the nighttime, elderly users should still wear the sensors because they may need to get up to have a drink or go to a restroom, for example. They feel very uncomfortable because the sensors around their necks may press on their chests or the bands may get wrapped around their necks when they turn over in their sleep. Therefore, it is better to wear the sensors around the neck during the daytime and on the wrist during the nighttime.

Taking fall detection of patients with a chronic disease as another example, the best solution for the patients who can walk is to wear the sensors around the neck; and the best solution for patients who lie in bed most of the time is to wear the sensors on the wrist. A fall detection system, which detects the fall only when the sensors are placed in one predefined position, cannot continue working if the patients' conditions change. For example, when the patients begin to walk frequently or they are not able to walk anymore, the system cannot continue working because the best positions of the sensors for the patients have changed. The patients have to pay for a new system for fall detection. Buying a new system adds to the patients' financial burdens, and it is also wasteful when the hardware of the old system is still in working order.

Considering the users' requirements mentioned above, it would be advantageous to enable the fall detection system to work when the sensors are placed in different positions on the body of the user. In addition, it would also be desirable that the fall detection system is easy to operate for the user.

To better address one or more of the above concerns, in a first aspect of the invention, a system for detecting a fall of a user is provided. The system comprises:

at least one sensor intended to be worn on the body of the user and configured to generate sensor data relating to the fall;

a determining unit configured to determine a sensor position of the at least one sensor; and

a processor configured to perform an analysis based on the sensor data and the sensor position to determine whether a fall is occurring or not.

Since the sensor position corresponds to a certain part of the body, the fall detection analysis performed by the processor is adjusted to match the movement characteristics of the certain part of the body. Therefore, the fall detection accuracy is guaranteed even if the sensor position changes. At the same time, the user feels good by wearing the sensors in their preferred way.

In an embodiment, the determining unit comprises a plurality of contacts configured to fasten a band for wearing the at least one sensor on the body, and a second circuit configured to detect a contact combination among the plurality of contacts, the contact combination corresponding to the sensor position among a plurality of predefined sensor positions.

By setting a plurality of contacts, the user is able to fasten the band with different contact combinations enabling the sensors to be worn on different parts of the body, and then the second circuit determines the sensor position by detecting the contact combination. Therefore, what the user is required to do is just to wear the sensors in different positions in different ways, and the system determines the sensor position automatically without any other extra actions from the user. So the whole process is very simple and the system is easy to operate for the user.

In a second aspect of the invention, a method of detecting a fall of a user is provided. The method comprises the steps of:

generating sensor data relating to the fall by at least one sensor intended to be worn on the body of the user;

determining a sensor position of the at least one sensor by a determining unit; and

performing an analysis based on the sensor data and the sensor position to determine by means of a processor whether a fall is occurring or not.

These and other aspects of the invention will be apparent from and elucidated with reference to the embodiments described hereinafter.

### DESCRIPTION OF THE DRAWINGS

The above and other objects and features of the present invention will become more apparent from the following detailed description considered in connection with the accompanying drawings, in which:

FIG. 1 shows a schematic diagram of an embodiment of the system according to the invention.

FIG. 2 (a) shows a schematic diagram of an embodiment of the determining unit; FIG. 2 (b) shows a schematic diagram of an embodiment of the plurality of contacts; FIG. 2 (c) shows a schematic diagram of an embodiment of the contact; FIG. 2 (d) shows a schematic diagram of an embodiment of the second circuit.

FIG. 3 is a flowchart showing a method in accordance with the invention.

The same reference numerals are used to denote similar parts throughout the figures.

### DETAILED DESCRIPTION

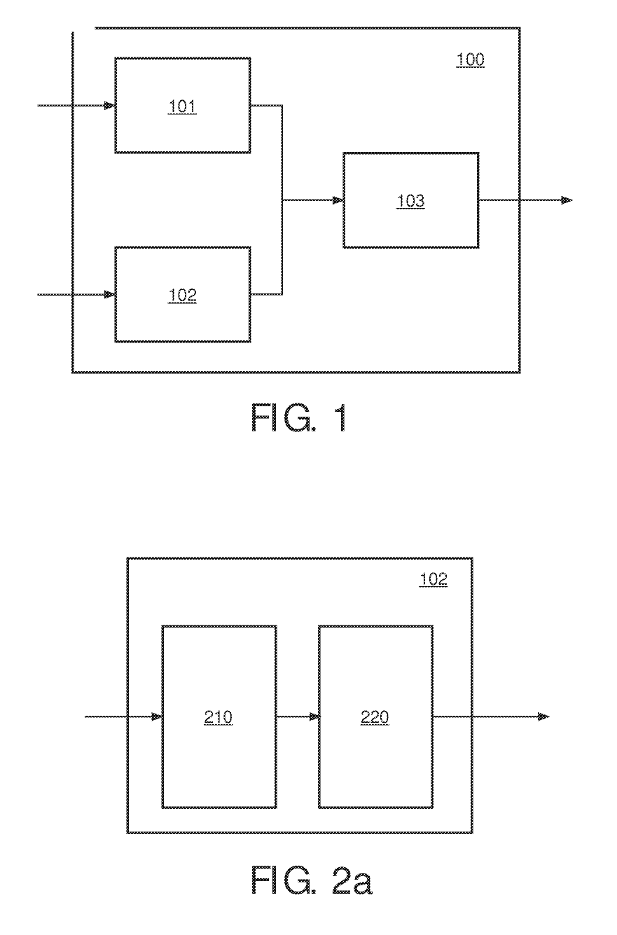

FIG. 1 shows a schematic diagram of an embodiment of the system 100 according to the invention.

The system 100 is intended for detecting a fall of a user. Referring to FIG. 1, the system 100 comprises at least one sensor 101 intended to be worn on the body of the user (not shown) and configured to generate sensor data relating to the fall. The system comprises one or more sensors 101; the sensors 101 can be the following sensors: an accelerometer, an altimeter, a thermometer and a clock.

Generally, the sensors 101 can be worn on the body via a holding fixture, such as a band, a clamp, etc. The sensors 101 can also be worn on the body by placing them in another portable device, such as a mobile phone, an mp3 player, etc. As the sensors 101 are worn on the body, the sensor data generated by the sensors 101 represents characteristics of the body movements, and thus can derive the body's status irrespective of whether the user moves or keeps still. The sensor data relating to a fall can be any one of acceleration, gravity, altitude, etc. Both the value and the direction of the acceleration and the gravity can be measured.

The system 100 further comprises a determining unit 102 configured to determine a sensor position of the at least one sensor 101. The sensors 101 can be worn on any part of the body, such as neck, waist, wrist, etc., as long as the sensor position is suitable for fall detection. The implementation of the determining unit will be elaborated later in detail.

The system 100 further comprises a processor 103 configured to perform an analysis based on the sensor data and the sensor position to determine whether a fall is occurring or not. The processor 103 exploits the sensor data and sensor position in many ways, for instance, the processor 103 adapts algorithms for fall detection according to the detected sensor position. For example, when the system 100 comprises an altimeter and an accelerometer, the sensor data including altitude, acceleration and gravity are measured. When the sensor position is the neck, the fall is determined when the acceleration increases suddenly and the gravity direction changes suddenly, and the altitude decreases more than one meter. When the sensor position is the wrist, gravity is not taken into consideration for fall detection, and the fall is determined when the altitude decreases more than fifty centimeters and subsequently the acceleration decreases suddenly. Therefore, the fall detection analysis is adjusted according to the sensor position and then a fall is detected based on the sensor data. A high detection accuracy is achieved while the user is able to wear the sensors 101 in their preferred way.

There are many ways to implement the determining unit 102.

In one embodiment, the determining unit 102 comprises an interface (not shown) configured to enable the user to select the sensor position from a plurality of predefined sensor positions. There are several possible positions on the body for wearing the sensors 101, and the plurality of predefined sensor positions can comprise all or part of said possible positions. For example, there are three possible positions for wearing the sensors, which are the neck, the waist and the wrist. The system 100 selects two of them, the neck and the wrist, as the predefined sensor positions. The interface may be built up in different ways, for example, a plurality of buttons for user interaction, and each button represents a predefined sensor position respectively, or a touch screen with a list of the plurality of predefined sensor positions for user selection, or a switch that enables the user to select a sensor position. In this way, the sensor position is easily and correctly set by the user and obtained by the determining unit 102.

In another embodiment, the determining unit 102 comprises a first circuit (not shown) configured to detect the length of a band 240 for wearing the at least one sensor 101 on the body, the length of the band 240 corresponding to the sensor position among a plurality of predefined sensor positions. There are many ways to detect the length of the band 240. For example, the length of the band 240 can be detected directly by measuring the resistance of the band 240 by the first circuit, if the band 240 is conductive, for example, made of metal. Since the length of the band 240 is detected for obtaining the sensor position, the sensor 101 can be worn in different positions on the body by using only one band 240 when the length of the band 240 is adjustable.

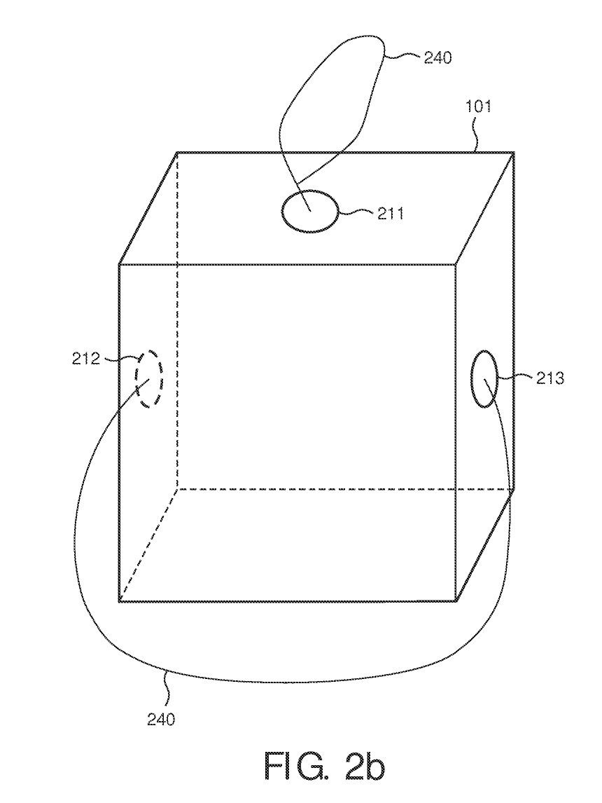

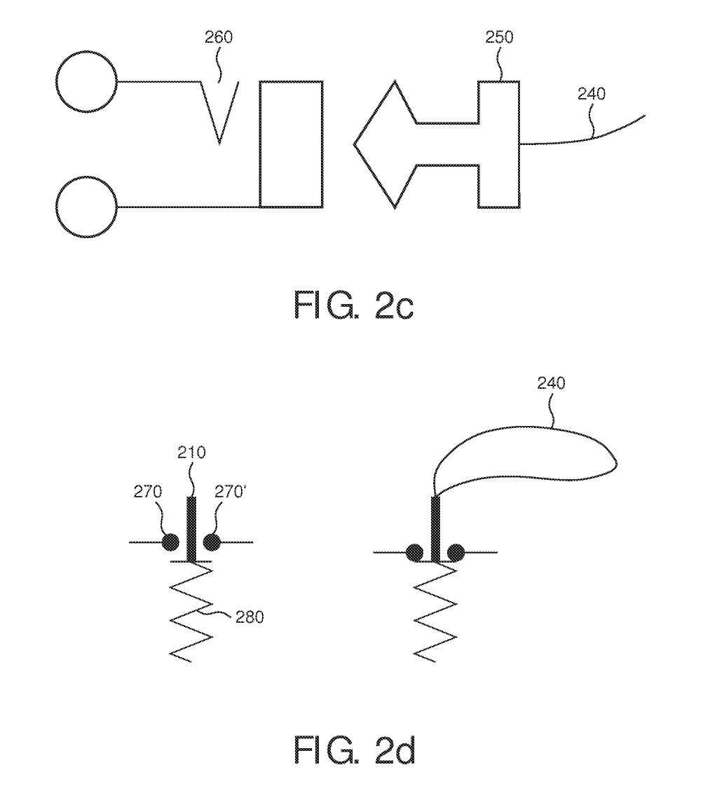

FIG. 2 (a) shows a schematic diagram of a further embodiment of the determining unit 102. FIG. 2 (b) shows a schematic diagram of an embodiment of the plurality of contacts 210. FIG. 2 (c) shows a schematic diagram of an embodiment of the contact 210. FIG. 2 (d) shows a schematic diagram of an embodiment of the second circuit 220.

Referring to FIG. 2 (a), a further embodiment of the determining unit 102 comprises a plurality of contacts 210 configured to fasten a band 240 for wearing the at least one sensor 101 on the body; and a second circuit 220 configured to detect a contact combination among the plurality of contacts 210, the contact combination corresponding to the sensor position among a plurality of predefined sensor positions.

It is possible to use one band 240 to wear the sensors 101 in any one of the plurality of predefined sensor positions or to use different bands 240 to wear the sensors 101 in different predefined sensor positions respectively. The number of bands 240 is equal or not equal to the number of the plurality of predefined sensor positions. The band 240 can be fastened to only a certain contact 210 or to several contacts 210 among the plurality of contacts 210. The band 240 is made of materials such as metal, plastic, cotton, chemical fiber, etc. In addition, the mapping between the different contacts 210 and the different bands 240 can be controlled by designing the contacts 210 in different sizes and shapes.

The contact combination is detected based on the contact number and/or contact distribution of the contact combination. In FIG. 2 (b), it is shown how to obtain the sensor position based on the contact number and/or contact distribution of the contact combination. There are two predefined sensor positions: the neck and the wrist of the body. In addition, there are three contacts 211, 212 and 213. Contact 211 is on the top side of the sensors 101 and contacts 212 and 213 are separately situated on the left and right sides of the sensors 101. One contact combination consisting of the contact 211 corresponds to the neck of the body, and another contact combination consisting of the contacts 212 and 213 corresponds to the wrist of the body. When the user places the sensors 101 around the neck, he fastens one band 240 to the contact 211 on the top side. The second circuit 220 detects that the contact number of the contact combination is one and the contact distribution of the contact combination is the top side, and then the sensor position is determined as the neck. When the user wears the sensors 101 on the wrist, he fastens another band 240 to the contacts 212 and 213 on the left and right sides. The second circuit 220 detects that the contact number of the contact combination is two and the contact distribution of the contact combination is the left and right sides, and then the sensor position is determined as the wrist. In the embodiment described above, the contact combination is determined based on both the contact number and contact distribution of the contact combination. It is also feasible to detect the contact combination only on the basis of the contact number or only on the basis of the contact distribution.

There are many ways to design the structure of the contact 210. FIG. 2 (c) illustrates one embodiment of the contact 210. The contact 210 comprises a jack 260 configured to mesh with a plug 250 situated on the band 240, and the second circuit 220 is configured to detect whether the jack 260 and the plug 250 are meshed.

The sensor position can be determined by carefully designing the second circuit 220. As shown in FIG. 2 (d), the second circuit 220 comprises a spring 280 and two open ends 270, 270′. The band 240 is fastened to the spring 280 through the contact 210. When the sensors 101 are worn on the wrist, the second circuit 220 is open as shown in the left part of FIG. 2 (d); when the sensors 101 are worn around the neck, the spring 280 stretches to touch the two open ends 270, 270′ so as to close the second circuit 220, as shown in the right part of FIG. 2 (d). Therefore, the second circuit 220 is able to determine the sensor positions irrespective of whether the sensors 101 are pendant or not, which corresponds to different positions.

The contacts 210 can be designed to make all possible contact combinations correspond to real sensor positions, or to make some possible contact combinations correspond to real sensor positions while the other possible contact combinations do not correspond to real sensor positions. In one embodiment, there are two contacts 210, two bands 240 and two predefined sensor positions, and the shapes of the two contacts 210 are different. In addition, one band 240 of the two bands 240 can be fastened to only one contact 210 of the two contacts 210, and each contact 210 corresponds to one sensor position. Therefore, the user cannot possibly mismatch the two bands 240 and the two contacts 210, and all possible contact combinations correspond to real sensor positions. In another embodiment, there are three contacts 210, one band 240 and two predefined sensor positions. In addition, the one band 240 can be fastened to any of the three contacts 210, and one contact 210 corresponds to one sensor position and the other two contacts 210 correspond to another sensor position. Therefore, the user may mismatch the one band 240 and the three contacts 210, and consequently some possible contact combinations correspond to real sensor positions and the other possible contact combinations do not correspond to real sensor positions.

The determining unit 102 is further configured to estimate a reference sensor position, based on the sensor data, and determine whether the sensor position is correct or not by comparing the sensor position with the reference sensor position, the sensor data being any one of, or a combination of, the following data: altitude, acceleration, gravity, temperature, and time.

The reference sensor position can be estimated in many ways. In one embodiment, a relatively low altitude indicates that the user is lying on his bed and the reference sensor position is estimated as the wrist, while a relatively high altitude indicates that the user is not lying on his bed and the reference sensor position is estimated as the neck. In another embodiment, the time after 21.00 hours indicates that the user is lying on his bed and the reference sensor position is estimated as the wrist, and the time before 21.00 hours indicates that the user is not lying on his bed and the reference sensor position is estimated as the neck. If the sensor position is the same as the reference sensor position, the sensor position is determined as being correct; otherwise, the sensor position is determined as not being correct.

The system 100 further comprises an output (not shown) configured to output a notifying message when the sensor position is not correct and/or output a warning message indicating a wrong contact combination when the sensor position determined by the contact combination is not a real sensor position.

The notifying message is output to remind the user that he may have wrongly operated the system 100. When the notifying message is output, the user checks whether he has operated the system 100 correctly or not and resets the system if there has been a wrong operation.

The warning message is output to remind the user that he has wrongly fastened the band 240 to the contacts 210, and the user is required to refasten the band 240 to the contacts 210.

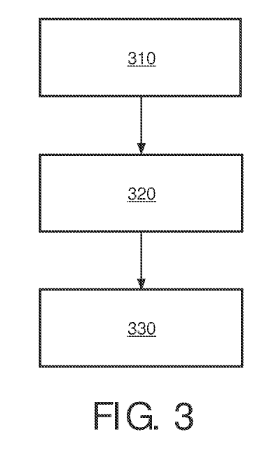

FIG. 3 is a flowchart showing a method in accordance with the invention.

With reference to FIG. 3, the method comprises a step 310 of generating sensor data relating to a fall by at least one sensor 101 intended to be worn on the body of the user.

The method further comprises a step 320 of determining a sensor position of the at least one sensor 101 by a determining unit 102.

The method further comprises a step 330 of performing an analysis based on the sensor data and the sensor position to determine whether a fall is occurring or not by a processor 103.

The sensors 101 provide the sensor data to the processor 103 either directly or with the support of a memory (not shown). The determining unit 102 provides the sensor position to the processor 103 directly or with the support of another memory (not shown).

The sensor position can be transmitted to a console (not shown) over a short distance or to a receiver (not shown), such as a mobile phone, over a long distance.

There are many ways to determine the sensor position of the at least one sensor 101.

In one embodiment, the step 320 of determining the sensor position comprises a sub-step of enabling the user to select the sensor position from a plurality of predefined sensor positions by means of an interface.

In another embodiment, the step 320 of determining the sensor position comprises a sub-step of detecting, by a first circuit, the length of a band 240 for wearing the at least one sensor 101 on the body, the length of the band 240 corresponding to the sensor position among a plurality of predefined sensor positions.

In a further embodiment, the step 320 of determining the sensor position comprises a sub-step of detecting a contact combination among a plurality of contacts 210 by a second circuit 220. The contact combination corresponds to the sensor position among a plurality of predefined sensor positions, and the plurality of contacts 210 is configured to fasten a band 240 for wearing the at least one sensor 101 on the body. It is the user who fastens the band 240 to the contacts 210.

In a still further embodiment, the step 320 of determining the sensor position comprises a sub-step of estimating a reference sensor position, based on the sensor data, and determining whether the sensor position is correct or not by comparing the sensor position with the reference sensor position by means of the determining unit 102, the sensor data being any one of, or a combination of, the following data: altitude, acceleration, gravity, temperature and time.

The method further comprises a step of outputting a notifying message by means of an output when the sensor position is not correct and/or outputting a warning message indicating a wrong contact combination by means of the output when the sensor position determined by the contact combination is not a real sensor position.

It should be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. In the systems claims enumerating several units, several of these units can be embodied by one and the same item of hardware or software. The usage of the words first, second and third, et cetera, does not indicate any ordering. These words are to be interpreted as names.

1. A system for detecting a fall of a user, the system comprising:
at least one sensor (101) intended to be worn on the body of the user and configured to generate sensor data relating to the fall;
a determining unit (102) configured to determine a sensor position of the at least one sensor (101); and
a processor (103) configured to perform an analysis based on the sensor data and the sensor position to determine whether a fall is occurring or not.

2. A system as claimed in claim 1, wherein the determining unit (102) comprises:
an interface configured to enable the user to select the sensor position from a plurality of predefined sensor positions.

3. A system as claimed in claim 1, wherein the determining unit (102) comprises:
a first circuit configured to detect the length of a band (240) for wearing the at least one sensor (101) on the body, the length of the band (240) corresponding to the sensor position among a plurality of predefined sensor positions.

4. A system as claimed in claim 1, wherein the determining unit (102) comprises:
a plurality of contacts (210) configured to fasten a band (240) for wearing the at least one sensor (101) on the body; and
a second circuit (220) configured to detect a contact combination among the plurality of contacts (210), the contact combination corresponding to the sensor position among a plurality of predefined sensor positions.

5. A system as claimed in claim 4, wherein the contact combination is detected based on the contact number and/or contact distribution of the contact combination.

6. A system as claimed in claim 4, wherein the contact (210) comprises a jack (260) configured to mesh with a plug (250) situated on the band (240), and the second circuit (220) is configured to detect whether the jack (260) and the plug (250) are meshed.

7. A system as claimed in claim 4, wherein the determining unit (102) is further configured to estimate a reference sensor position based on the sensor data and determine whether the sensor position is correct or not by comparing the sensor position with the reference sensor position, the sensor data being any one of, or a combination of, the following data: altitude, acceleration, gravity, temperature and time.

8. A system as claimed in claim 7, further comprising an output configured to output a notifying message when the sensor position is not correct and/or output a warning message indicating a wrong contact combination when the sensor position determined by the contact combination is not a real sensor position.

9. A method of detecting a fall of a user, the method comprising the steps of:
generating (310) sensor data relating to the fall by at least one sensor (101) intended to be worn on the body of the user;
determining (320) a sensor position of the at least one sensor (101) by a determining unit (102); and
performing (330) an analysis based on the sensor data and the sensor position to determine whether a fall is occurring or not by means of a processor (103).

10. A method as claimed in claim 9, wherein the step of determining (320) comprises a sub-step of:
enabling the user to select the sensor position from a plurality of predefined sensor positions by means of an interface.

11. A method as claimed in claim 9, wherein the step of determining (320) comprises a sub-step of:
detecting the length of a band (240) for wearing the at least one sensor (101) on the body by means of a first circuit (321), the length of the band (240) corresponding to the sensor position among a plurality of predefined sensor positions.

12. A method as claimed in claim 9, wherein the step of determining (320) comprises a sub-step of:
detecting a contact combination among a plurality of contacts (210) by means of a second circuit (220), the contact combination corresponding to the sensor position among a plurality of predefined sensor positions, and the plurality of contacts (210) being configured to fasten a band (240) for wearing the at least one sensor (101) on the body.

13. A method as claimed in claim 12, wherein the step of determining (320) comprises a sub-step of:
estimating a reference sensor position based on the sensor data and determining whether the sensor position is correct or not by comparing the sensor position with the reference sensor position by means of the determining unit (102), the sensor data being any one of, or a combination of, the following data: altitude, acceleration, gravity, temperature, and time.

14. A method as claimed in claim 13, further comprising a step of:
outputting a notifying message by an output when the sensor position is not correct and/or outputting a warning message indicating a wrong contact combination by the output when the sensor position determined by the contact combination is not a real sensor position.
